(19) United States
(12) Patent Application Publication
(10) Pub. No.: US 2009/0082408 A1
(43) Pub. Date: Mar. 26, 2009

(54) NOVEL HETEROCYCLIC DERIVATIVES

(76) Inventors: Surendrakumar Satyanarayan Pandey, Tamil Nadu (IN); Gajendra Singh, Tamil Nadu (IN); Kadnur Venkatachalapathi Sanjay, Tamil Nadu (IN); Bishwajit Nag, Union City, CA (US)

Correspondence Address: LADAS & PARRY LLP, 224 SOUTH MICHIGAN AVENUE, SUITE 1600, CHICAGO, IL, 60604, US

(21) Appl. No.: 11/914,484

(22) PCT Filed: May 18, 2006
(86) PCT No.: PCT/IB2006/001304

§ 371 Date: May 20, 2008

(30) Foreign Application Priority Data
May 19, 2005 (IN) 597/CHE/2005

(51) Int. Cl. A61K 31/4402; C07D 211/72; A61P 35/00

(52) U.S. Cl. 514/352; 546/312

(57) ABSTRACT

The present invention relates to novel heterocyclic derivatives of the general formula (I), their pharmaceutically acceptable salts, and their pharmaceutical compositions. The present invention more particularly provides novel compounds of the general formula (I).

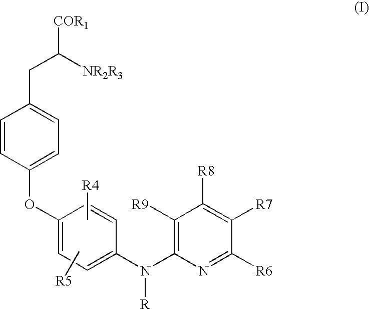

The following specification particularly describes the nature of the invention and the manner in which it has to be performed;

### FIELD OF THE INVENTION

The present invention relates to novel heterocyclic derivatives of the general formula (I), their pharmaceutically acceptable salts, and their pharmaceutical compositions. The present invention more particularly provides novel compounds of the general formula (I).

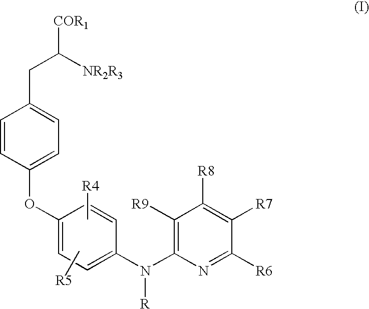

The present invention also relates to a process for the preparation of the above said novel compounds and compositions containing them.

The compounds of the present invention are effective in lowering blood glucose, serum insulin, free fatty acids, cholesterol, triglyceride levels and are useful in the treatment and/or prophylaxis of type II diabetes. These compounds are effective in the treatment of obesity, inflammation, autoimmune diseases such as multiple sclerosis and rheumatoid arthritis. Surprisingly, these compounds increase the leptin level and have no liver toxicity.

Furthermore, the compounds of the present invention are useful for the treatment of disorders associated with insulin resistance, such as polycystic ovary syndrome, as well as hyperlipidemia, coronary artery disease and peripheral vascular disease.

### BACKGROUND OF THE INVENTION

The causes of type I and II diabetes are not yet clear, although both genetics and environment seem to be the factors. Type I diabetes is an autonomic immune disease and the patient must take insulin to survive. Type II diabetes is the more common form, it is a metabolic disorder resulting from the body's inability to make a sufficient amount of insulin or to properly use the insulin that is produced. Insulin secretion and insulin resistance are considered to be the major defects, however, the precise genetic factors involved in the mechanism remain unknown.

Patients with diabetes usually have one or more of the following defects:
Less production of insulin by the pancreas;
Over secretion of glucose by the liver;
Independence of the glucose uptake by the skeletal muscles;
Defects in glucose transporters, desensitization of insulin receptors;
Defects in the metabolic breakdown of polysaccharides.

Other than the parenteral or subcutaneous administration of insulin, there are about 4 classes of oral hypoglycemic agents used i.e. sulfonylurea, biguanides, alpha glucosidase inhibitors and thiazolidinediones. Each of the current agents available for use in the treatment of diabetes has certain disadvantages. Accordingly, there is a continuing interest in the identification and development of new agents, which can be orally administered, for use in the treatment of diabetes.

The thiazolidinedione class listed above has gained more widespread use in the recent years for the treatment of type II diabetes, exhibiting particular usefulness as insulin sensitizers to combat “insulin resistance”, a condition in which the patient becomes less responsive to the effects of insulin. However, there is a continuing need for nontoxic, more widely effective insulin sensitizers. In our continuous efforts to explore new compounds having antidiabetic activity, we propose to synthesize new compounds containing heterocyclic rings namely the substituted pyridine ring.

Few Prior Art Reference which Disclose the Closest Compounds are Given Here:
I) US patent 2004/0142991 discloses compounds of the formula (I)

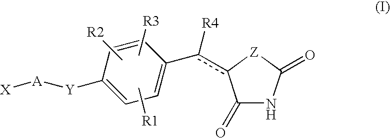

wherein ---- represents optional double bond; Y represents oxygen, sulfur or NR, wherein R represents hydrogen or alkyl; Z represents oxygen or sulfur; R₁, R₂, R₃ and R₄ may be same or different and independently represent hydrogen, halogen, hydroxy, nitro, cyano, formyl, amino, alkyl, alkoxy group; A represents a bond or substituted or unsubstituted aryl, heterocyclyl or heteroaryl ring; X represents amino acid or its derivatives
Exemplified below is a compound of this formula (I)

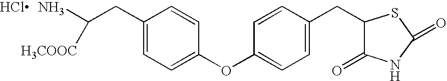

II) WO 93/00337 discloses compounds of formula (I) in the treatment of diabetes that have useful pharmacological properties, producing an action on the intermediate metabolism and in particular, lowering of blood-sugar levels.

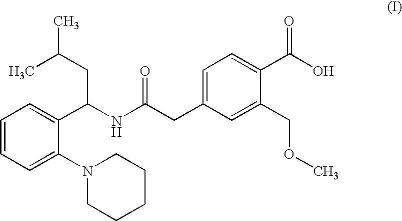

III) U.S. Pat. No. 4,572,912 discloses compounds of formula (I) and a series of new thiazolidine derivatives which likewise have the ability to lower blood lipid and blood sugar levels.

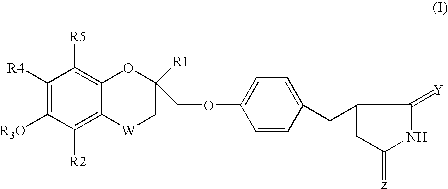

R₁ and R₂ are the same or different and each represents hydrogen or C₁-C₅ alkyl; R₃ represents hydrogen, an acyl group, a (C₁-C₆ alkoxy) carbonyl group or an aralkyloxycarbonyl group; R₄ and R₅ are the same or different and each represents hydrogen, C₁-C₅ alkyl or C₁-C₅ alkoxy, or R₄ and R₅ together represent a C₁-C₄ alkylenedioxy group; n is 1, 2 or 3; W represents the —CH₂—, >CO or >CH—OR₆ group (in which R₆ represents any one of the atoms or groups defined for R₃ and may be the same as or different from R₃); Y and Z are the same or different and each represents oxygen or imino.
IV) U.S. Pat. No. 4,687,777 discloses thiazolidinedione derivatives of the formula (I) and their pharmacologically acceptable salts as novel compounds which exhibit in mammals, a blood sugar and lipid-lowering activity, and which are of value as therapeutic agents for the treatment of diabetes and hyperlipemia.

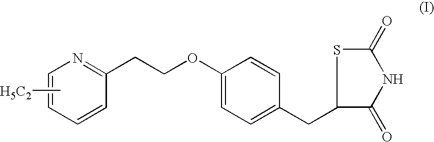

### OBJECTIVE OF THE INVENTION

With an objective to develop novel compounds for lowering blood glucose, free fatty acids, cholesterol and triglyceride levels in the type II diabetes and to treat autoimmune diseases such as multiple sclerosis and rheumatoid arthritis we focused our research to develop new compounds effective in the treatment of the above mentioned diseases and efforts in this direction have led to compounds having the general formula (I).

The main objective of the present invention is therefore, to provide novel heterocyclic derivatives and their pharmaceutically acceptable salts that are also useful for the treatment of disorders associated with insulin resistance, such as polycystic ovary syndrome, as well as hyperlipidemia, coronary artery disease and peripheral vascular disease. Another objective of the present invention is to provide novel heterocyclic derivatives and their pharmaceutically acceptable salts having enhanced activities, without toxic effects or with reduced toxic effects. Yet another objective of the present invention is to provide a process for the preparation of novel heterocyclic derivatives of the formula (I) and their pharmaceutically acceptable salts.

### SUMMARY OF THE INVENTION

The present invention relates to novel heterocyclic derivatives of the general formula (I)

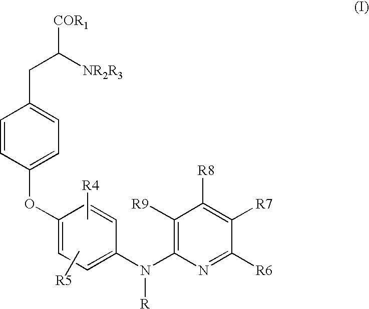

their pharmaceutically acceptable salts, and their pharmaceutical compositions; wherein R represents hydrogen, C₁-C₄ alkyl, aryl groups such as phenyl, naphthyl and the like; R₁ represents —OR¹⁰ where R¹⁰ represents hydrogen, substituted or unsubstituted groups selected from C₁-C₄ alkyl or a counter ion, NR¹¹R¹², where R¹¹ and R¹² may be same or different and independently represent H, substituted or unsubstituted groups selected from C₁-C₄ alkyl, aryl groups such as phenyl, naphthyl and the like, heteroaryl groups; R₂ and R₃ may be same or different and independently represent H, COR¹³, substituted or unsubstituted groups selected from C₁-C₄ allyl; where R¹³ represents substituted or unsubstituted groups selected from C₁-C₄ alkyl, aryl groups such as phenyl, naphthyl and the like, heteroaryl, aryloxy, alkoxy or aralkoxy groups; R₄ and R₅, may be same or different and independently represent hydrogen, halogen, hydroxy, nitro, cyano, formyl, amino, C₁-C₄ alkyl, haloalkyl, alkoxy groups; R₆, R₇, R₈ and R₉ may be same or different and independently represents hydrogen, nitro, cyano, hydroxy, formyl, azido, halo, or substituted or unsubstituted groups selected from C₁-C₄ alkyl, alkoxy, acyl, haloalkyl, amino, hydrazine, monoalkylamino, dialkylamino, acylamino, alkylsulfonyl, alkylsulfinyl, arylsulfonyl, arylsulfinyl, alkylthio, arylthio, alkoxycarbonyl, aryloxycarbonyl, alkoxyalkyl, sulfamoyl, carboxylic acid or its derivatives.

### DETAILED DESCRIPTION OF THE INVENTION

Suitable groups represented by R represents hydrogen, substituted or unsubstituted linear or branched C₁-C₄ alkyl groups such as methyl, ethyl, n-propyl, isopropyl, n-butyl, isobutyl, t-butyl and the like; aryl groups such as phenyl, naphthyl and the like, the aryl group may be substituted;

Suitable groups represented by R₁ represent —OR¹⁰, NR¹¹R¹²;

Suitable groups represented by R₂ and R₃ are selected from H, COR¹³, substituted or unsubstituted linear or branched C₁-C₄ alkyl groups such as methyl, ethyl, n-propyl, isopropyl, n-butyl, isobutyl, t-butyl and the like;

Suitable groups represented by R₄ and R₅ are selected from hydrogen, halogen atoms such as fluorine, chlorine, bromine or iodine; hydroxy, nitro, cyano, formyl, amino, unsubstituted linear or branched C₁-C₄ alkyl groups such as methyl, ethyl, n-propyl, isopropyl, n-butyl, isobutyl, t-butyl and the like; haloalkyl groups such as chloromethyl, chloroethyl, trifluoromethyl, trifluoroethyl, dichloromethyl, dichloroethyl, trichloromethyl, difluoromethyl, and the like, which may be substituted; alkoxy groups such as methoxy, ethoxy, n-propoxy, isopropoxy and the like, which may be substituted; R₆, R₇, R₈ and R₉ may be same or different and are selected from hydrogen, halogen atoms such as fluorine, chlorine, bromine or iodine; hydroxy, nitro, cyano, formyl, amino, azido, hydrazine; unsubstituted or unsubstituted groups selected from linear or branched C₁-C₄ allyl group such as methyl, ethyl, n-propyl, isopropyl, n-butyl, isobutyl, t-butyl and the like; haloalkyl groups such as chloromethyl, chloroethyl, trifluoromethyl, trifluoroethyl, dichloromethyl, dichloroethyl, trichloromethyl, difluoromethyl, and the like, which may be substituted; alkoxy groups such as methoxy, ethoxy, n-propoxy, isopropoxy and the like, which may be substituted; monoalkylamino groups such as —NHCH₃, —NHC₂H₅, —NHC₃H₇, —NHC₆H₁₃, and the like, which may be substituted; dialkylamino groups such as —N(CH₃)₂, —NCH₃(C₂H₅), —N(C₂H₅)₂ and the like, which may be substituted; carboxylic acids or its derivatives such as esters or amides; acylamino group such as —NHC(═O)CH₃, —NHC(═O)C₂H₅, —NHC(—O)C₃H₇, —NHC(═O)C₆H₁₃, and the like, which may be substituted; alkylsulfonyl groups such as methylsulfonyl, ethylsulfonyl, n-propylsulfonyl, iso-propylsulfonyl and the like, the alkylsulfonyl group may be substituted; arylsulfonyl groups such as phenylsulfonyl or naphthylsulfonyl, the arylsulfonyl group may be substituted; alkylsulfinyl groups such as methylsulfinyl, ethylsulfinyl, n-propylsulfinyl, iso-propylsulfinyl and the like, the alkylsulfinyl group may be substituted; arylsulfinyl groups such as phenylsulfinyl or naphthylsulfinyl, the arylsulfinyl group may be substituted; alkylthio groups such as methylthio, ethylthio, n-propylthio, iso-propylthio and the like, the alkylthio group may be substituted; alkoxycarbonyl groups such as methoxycarbonyl, ethoxycarbonyl, n-propoxycarbonyl, isopropoxycarbonyl and the like, the alkoxycarbonyl group may be substituted; aryloxycarbonyl groups such as phenoxycarbonyl, naphthoxycarbonyl, the aryloxycarbonyl group may be substituted; alkoxyalkyl groups such as methoxymethyl, ethoxymethyl, methoxyethyl, ethoxyethyl and the like, which may be substituted; sulfamoyl, carboxylic acid or its derivatives.

Suitable groups represented by R¹⁰ are selected from hydrogen, substituted or unsubstituted linear or branched C₁-C₄ alkyl groups such as methyl, ethyl, n-propyl, isopropyl, n-butyl, isobutyl, t-butyl and the like; the counter ion is selected from alkali metals like Li, Na, and K; alkaline earth metals like Ca and Mg; salts of different bases such as ammonium or substituted ammonium salts, diethanolamine, α-phenylethylamine, benzylamine, piperidine, morpholine, pyridine, hydroxyethylpyrrolidine, hydroxyethylpiperidine, choline and the like, aluminum, tromethamine and the like.

Suitable groups represented by R¹¹ and R¹² are selected from hydrogen, substituted or unsubstituted linear or branched C₁-C₄ alkyl groups such as methyl, ethyl, n-propyl, isopropyl, n-butyl, isobutyl, t-butyl and the like; aryl groups such as phenyl, naphthyl and the like, the aryl group may be substituted; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl, quinolinyl and the like;

Suitable groups represented by R¹³ are selected from hydrogen, substituted or unsubstituted linear or branched C₁-C₄ alkyl groups such as methyl, ethyl, n-propyl, isopropyl, n-butyl, isobutyl, t-butyl and the like; aryl groups such as phenyl, naphthyl and the like, the aryl group may be substituted; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl, quinolinyl and the like; alkoxy groups such as methoxy, ethoxy, n-propoxy, isopropoxy and the like, which may be substituted; aralkoxy groups such as phenylmethoxy, phenylethoxy, phenylpropoxy, and the like; aryloxy groups such as phenoxy, naphthoxy and the like

Suitable substituents on the groups represented by R₁, R₂, R₃ R₄, R₅, R₆ R₇, R₈, R₉ are selected from nitro, hydroxy, halo, formyl, azido, alkyl, alkoxy, acyl, haloalkyl, amino, hydrazine, monoalkylamino, dialkylamino, acylamino, alkylsulfonyl, alkylsulfinyl, arylsulfonyl, arylsulfinyl, alkylthio, arylthio, alkoxycarbonyl, aryloxycarbonyl, alkoxyalkyl, sulfamoyl, carboxylic acid or its derivatives.

Pharmaceutically acceptable salts of the present invention include alkali metal like Li, Na, and K, alkaline earth metal like Ca and Mg, salts of organic bases such as diethanolamine, α-phenylethylamine, benzylamine, piperidine, morpholine, pyridine, hydroxyethylpyrrolidine, hydroxyethylpiperidine, choline and the like, ammonium or substituted ammonium salts, aluminum salts. Salts also include amino acid salts such as glycine, alanine, cystine, cysteine, lysine, arginine, phenylalanine, guanidine etc. Salts may include acid addition salts where appropriate which are sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Pharmaceutically acceptable solvates may be hydrates or comprising other solvents of crystallization such as alcohols.

Particularly useful compounds according to the invention include:
Methyl-2-amino-3-{4-[4-(pyridin-2-ylamino)phenoxy]phenyl}propanoate dihydrochloride;
Methyl(2S)-2-amino-3-{4-[4-(pyridin-2-ylamino)phenoxy]phenyl}propanoate dihydrochloride;
Methyl (2S)-2-amino-3-(4-{4-[(5-nitropyridin-2-yl)amino]phenoxy}phenyl)propanoate dihydrochloride;
Methyl (2S)-2-amino-3-(4-{4-[(3-cyanopyridin-2-yl)amino]phenoxy}phenyl) propanoate dihydrochloride;
Methyl (2S)-2-amino-3-(4-{4-[(5-trifluoromethyl)pyridin-2-yl)amino]phenoxy}phenyl)propanoate dihydrochloride;
Methyl (2S)-2-amino-3-(4-{4-[(3-nitropyridin-2-yl)amino]phenoxy}phenyl)propanoate dihydrochloride;
Methyl (2S)-2-amino-3-(4-{2-fluoro-4-[(5-nitropyridin-2-yl)amino]phenoxy}phenyl)propanoate dihydrochloride;
Methyl (2S)-2-amino-3-(4-{2-fluoro-4-[(5-trifluoromethyl)pyridin-2-yl)amino]phenoxy}phenyl)propanoate dihydrochloride;
Methyl (2S)-2-amino-3-(4-{2-fluoro-4-[(3-nitropyridin-2-yl)amino]phenoxy}phenyl)propanoate dihydrochloride;
(2S)-2-Amino-3-{4-[4-(pyridin-2-ylamino)phenoxy]phenyl}propanoic acid dihydrochloride;

Preferred salts for the list of compounds given above are hydrochloride, hydrobromide, sodium, potassium or magnesium.

In another aspect the invention provides novel pharmaceutical compositions comprising the heterocyclic derivatives of formula (I) as set out above. The said compositions may comprise the heterocyclic derivatives as active ingredient together with pharmaceutically acceptable carrier, diluent or excipient. The composition may be prepared by processes known in the art and may be in the form of a tablet, capsule, powder, syrup, solution or suspension. The amount of active ingredient in the composition may be less than 60% by weight.

According to another feature of the present invention, there is provided a process for the preparation of compounds of the formula (I), wherein all other symbols are as defined earlier, as shown in the scheme-I

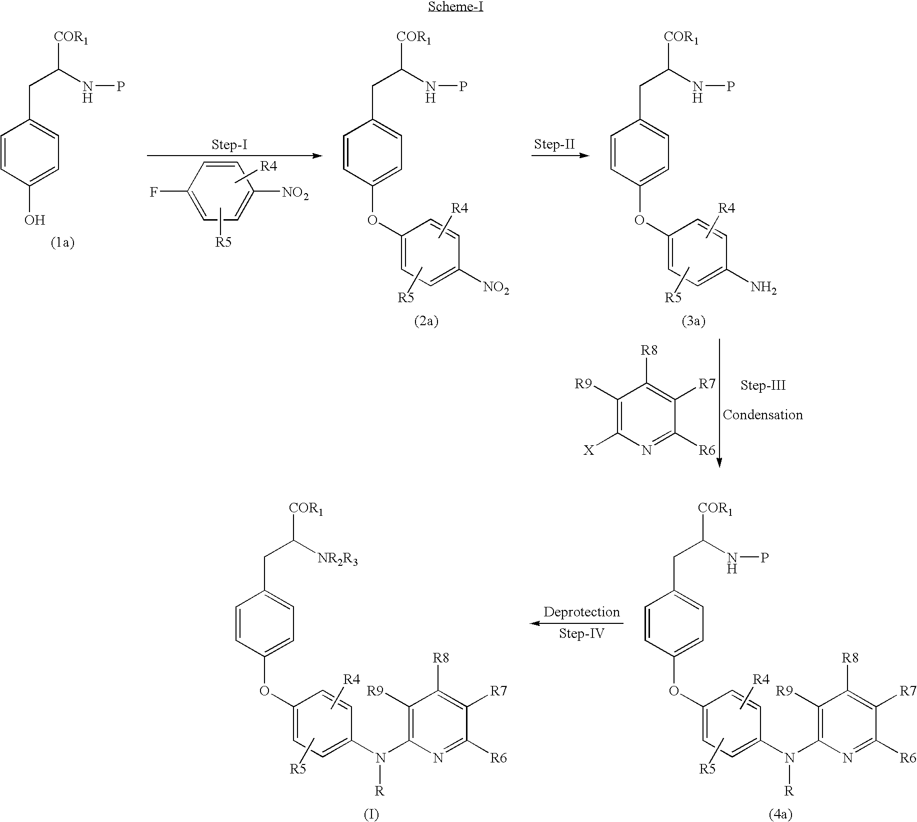

The compounds of the general formula (I) are prepared by the following procedure;

Step-I: Condensation of the amino acid derivative of the compound of formula (1a), (wherein P represents a protecting group) with halo nitro benzene carried out in the presence of solvents selected from toluene, DMF, tetrahydrofuran, chloroform, dichloromethane, dichloroethane, ethyl acetate, o-dichlorobenzene or a mixture thereof, in the presence of a base such as triethyl amine, diethylamine, pyridine, DMAP, alkali hydroxides, alkaline earth metal hydroxide, alkali carbonates such as sodium hydroxide, potassium hydroxide, potassium carbonate and the like gave the compound of the formula (2a). The reaction is carried out at a temperature in the range of room temperature to reflux temperature, mostly 0° C. to 100° C. Alternatively the single S isomer of the compound of formula (2a) is prepared by condensation of the compound of formula (1a) (wherein R₁ is OH) with halo nitro benzene followed by alkylation by conventional methods.

Step-II: Hydrogenation of the compound of the formula (2a) by using a catalyst such as Raney nickel, Pd/C in the presence of solvents such as, methanol, ethanol, ethylacetate, n-butylacetate or a mixture thereof. The reaction may be carried out at 0° C. to 100° C. and the duration of the reaction may range from 2 to 24 hours, to produce a compound of the formula (3a).

Step-III: The compound of formula (3a) is reacted with halo pyridines in the presence of solvents such as toluene, methanol, ethanol, tetrahydrofuran, chloroform, dichloromethane, dichloroethane, ethylacetate, o-dichlorobenzene or a mixture thereof or without solvent. The reaction may be carried out at 50° C. to 150° C. and the duration of the reaction may range from 2 to 24 hours, to produce a compound of the formula (4a).

Step-IV: Deprotection of compound of formula (4a) may be carried out using Pd/C or HCl in the presence of solvents. Alternatively the deprotection may also be carried out by passing HCl gas in the presence of solvents selected from acetonitrile, dichloromethane, methanol, dimethylsulfoxide, dimethylformamide, tetrahydrofuran, trifluoroacetic acid, 1-methyl-2-pyrrolidinone, N,N-dimethylacetamide and the like or mixtures thereof.

It is appreciated that in any of the above-mentioned reactions, any reactive group in the substrate molecule may be protected according to the conventional chemical practice. Suitable protecting groups in any of the above-mentioned reactions are those used conventionally in the art. The methods of formation and removal of such protecting groups are those conventional methods appropriate to the molecule being protected. More specifically the protecting groups P used particularly in the present invention are conventional protecting groups such as t-butoxy carbonyl (t-Boc), trityl, trifluoroacetyl, benzyloxy, benzyloxy carbonyl (Cbz) and the like and deprotection can be done by conventional methods.

The pharmaceutically acceptable salts are prepared by reacting the compound of formula (I) with 1 to 4 equivalents of a base such as sodium hydroxide, sodium methoxide, sodium hydride, potassium t-butoxide, calcium hydroxide, magnesium hydroxide and the like, in solvents like ether, THF, methanol, t-butanol, dioxane, isopropanol, ethanol etc. Mixtures of solvents may be used. Organic bases like lysine, arginine, diethanolamine, choline, guanidine and their derivatives etc. may also be used. Alternatively, acid addition salts are prepared by treatment with acids such as hydrochloric acid, hydrobromic acid, nitric acid, sulfuric acid, phosphoric acid, p-toluenesulfonic acid, methanesulfonic acid, acetic acid, citric acid, maleic acid, salicylic acid, hydroxynaphthoic acid, ascorbic acid, palmitic acid, succinic acid, benzoic acid, benzene sulfonic acid, tartaric acid and the like in solvents like ethyl acetate, ether, alcohols, acetone, THF, dioxane etc. Mixture of solvents may also be used.

The invention is explained in details in the examples given below which are provided by the way of illustration only and therefore should not be construed to limit the scope of the invention.

### EXAMPLE 1

### Preparation of Methyl-2-amino-3-{4-[4-(pyridin-2-ylamino)phenoxy]phenyl}propionate dihydrochloride

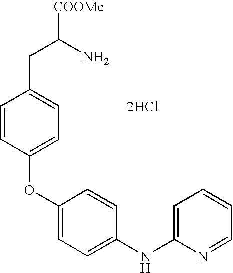

### Step: I

### Synthesis of Methyl 2-[(tert-butoxycarbonyl)amino]-3-[4-(4-nitrophenoxy)phenyl]propanoate

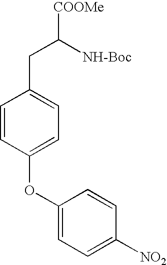

To a solution of boc-tyrosine-methyl ester (10 g, 33.8 mmol) and potassium carbonate (23.3 g, 169.4 mmol) in dimethylformamide (70 ml) was charged 4-fluoro nitrobenzene (9.5 g, 67.7 mmol). The reaction mixture was heated to 70° C. for 15 hours subsequently it was quenched with saturated cold ammonium chloride solution (300 ml) and extracted with ethyl acetate. The solvent was evaporated to give the desired product 11 g (78%), ¹HNMR [CDCl3, 400 MHz] δ ppm: 1.42 (s, 9H), 3.04 (m, 1H)), 3.15 (m, 1H), 3.74 (s, 3H), 4.6 (m, 1H), 5.03 (d, 1H), 7.00 (m, 4H), 7.19 (m, 2H), 8.19 (m, 2H); m/z^{M+1} 417.1

### Step: II

### Synthesis of Methyl 2-[(tert-butoxycarbonyl)amino]-3-[4-(4-aminophenoxy)phenyl]propanoate

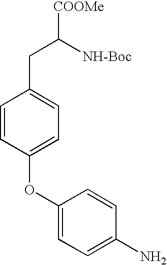

10% Pd/C (0.6 g) was added to a solution of methyl 2-amino-3-[4-(4-nitrophenoxy)phenyl]propanoate (6.19 g, 14.6 mmol) in dichloromethane (300 ml) and hydrogenated at 30 psi for 11 hours. After completion of reaction the catalyst was filtered off, and the reaction mixture was concentrated to give methyl 2-amino-3-[4-(4-aminophenoxy)phenyl]propanoate 5.5 g (97.1%); ¹HNMR [DMSO-d₆, 400 MHz] δ ppm: 1.30 (s, 9H), 2.75 (m, 1H), 2.88 (m, 1H), 3.58 (s, 3H), 4.2 (m, 1H), 4.96 (bs, 2H), 6.54 (d, 2H), 6.71 (m, 4H), 7.13 (d, 2H), 7.27 (d, 1H); m/z^{M-1} 387.2

### Step: III

### Synthesis of Methyl 2-[(tert-butoxycarbonyl)amino]-3-{4-[4-(pyridin-2-ylamino) phenoxy]phenyl}propanoate

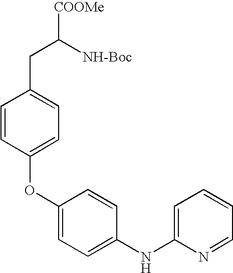

Methyl 2-[(tert-butoxycarbonyl)amino]-3-[4-(4-aminophenoxy)phenyl]propanoate (0.6 g, 1.55 mmol) and 2-chloro pyridine (1.2 ml, 10.57 mmol) were stirred under a nitrogen atmosphere at 130° C. for 20 hours. After completion of the reaction, the reaction mixture was quenched with ammonium chloride solution (25 ml) and was extracted with ethylacetate (3×25 ml). The solvent was evaporated to give the crude product, which was purified by column chromatography to yield the desired product (0.41 g, 61.5%) ¹HNMR. [CDCl3, 400 MHz] δ ppm: 1.42 (s, 9H), 3.0 (m, 2H), 3.72 (s, 3H), 4.57 (m, 1H), 5.0 (d, 1H), 6.51 (s, 1H), 6.72 (m, 1H), 6.77 (d, 1H), 6.92 (d, 2H), 6.99 (d, 2H), 7.07 (d, 2H), 7.31 (d, 2H), 7.47 (t, 1H), and 8.17 (d, 1H); m/z^{M+1} 464.2

### Step: IV

### Synthesis of Methyl 2-amino-3-{4-[4-(pyridin-2-ylamino)phenoxy]phenyl}propanoate dihydrochloride

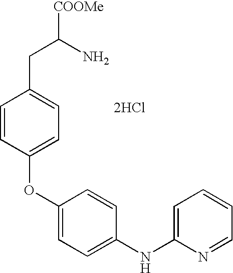

To a solution of methyl 2-[(tert-butoxycarbonyl)amino]-3-{4-[4-(pyridin-2-ylamino)phenoxy]phenyl}propanoate (0.29 g, 0.43 mmol) in dichloromethane (25 ml) was bubbled dry HCl gas for 2 hours. After completion of the reaction, the excess of HCl gas was removed by nitrogen gas bubbling and the solvent was removed under reduced pressure to give the product as an off white solid 0.100 g (66%), ¹HNMR. [DMSO-d₆, 400 MHz] δ ppm: 3.1 (dd, 2H), 3.7 (s, 3H), 4.2 (m, 1H), 6.9 (t, 1H), 7.0 (d, 2H), 7.07 (dd, 3H), 7.2 (d, 2H), 7.5 (d, 2H), 8.0 (d, 1H), 8.1 (d, 1H), 8.5 (bs, 2H), 10.31 (bs, 1H); m/z^{M+1} 363.9

### EXAMPLE 2

### Methyl (2S)-2-amino-3-{4-[4-(pyridin-2-ylamino)phenoxy]phenyl}propanoate dihydrochloride

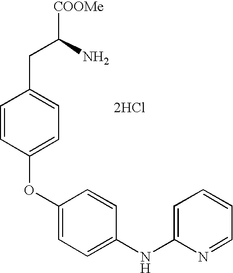

### Step: I

### Synthesis of 2-[(tert-butoxycarbonyl)amino]-3-[4-(4-nitrophenoxy)phenyl]propanoic acid

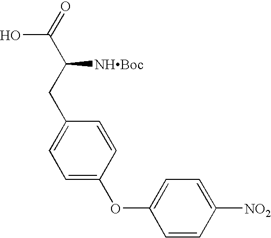

To a solution of N-tert-butoxy carbonyl-L-tyrosine (10 g, 35.5 mmol) and potassium carbonate (29.4 g, 213.5 mmol) in dimethylformamide (50 ml) was charged 4-fluoro nitrobenzene (6.02 g, 43.6 mmol). The reaction mixture was heated to 80° C. for 15 hours. After completion of the reaction, the reaction mixture was quenched with saturated cold ammonium chloride solution (300 ml), extracted with ethyl acetate and separated off the organic layer. The aqueous layer was acidified using 2N HCl to pH 2 and was extracted with ethyl acetate. The combined organic layer was washed with brine, dried over anhydrous sodium sulphate and concentrated under reduced pressure to yield the product 14.13 g (98.5%), ¹HNMR [CDCl3, 400 MHz] δ ppm: 1.39 (s, 9H), 2.92 (m, 1H)), 3.0 (d, 1H), 4.35 (m, 1H), 7.06 (m, 4H), 7.32 (m, 2H), 8.22 (m, 2H); m/z^{M+1} 403.2

### Step: II

### Synthesis of 2-[(tert-butoxycarbonyl)amino]-3-[4-(4-nitrophenoxy)phenyl]propanoic acid methyl ester

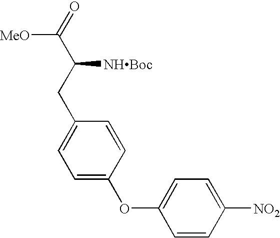

To a solution of 2-[(tert-butoxycarbonyl)amino]-3-[4-(4-nitrophenoxy)phenyl]propanoic acid and sodium bicarbonate (4.38 g, 52.25 mmol) in dry DMF (40 ml), was added iodomethane (14.83 g, 104.4 mmol) under an inert atmosphere and the reaction mixture was stirred at room temperature for 6 hours. After completion of the reaction, the reaction mixture was quenched with 0.5M KOH solution and was extracted with ethyl acetate. The organic layer was washed with brine and evaporated under reduced pressure to yield the desired product 7.0 g (97.2%), ¹HNMR [CDCl3, 400 MHz] δ ppm: 1.42 (s, 9H), 3.15 (m, 1H)), 3.18 (m, 1H), 3.74 (s, 3H), 4.6 (m, 1H), 5.06 (m, 1H), 7.0 (m, 4H), 7.19 (m, 2H), 8.19 (m, 2H); m/z^{M+1} 417.2

### Step: III

### Synthesis of methyl 2-[(tert-butoxycarbonyl)amino]-3-[4-(4-aminophenoxy)phenyl]propanoate (prepared according to the procedure given in the Example 1, step II)

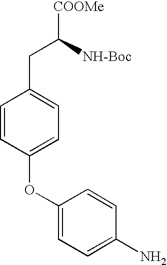

Yield (5.48 g, 84.4%); ¹HNMR [CDCl₃, 400 MHz] δ ppm: 1.41 (s, 9H), 3.02 (m, 2H), 3.60 (bs, 2H), 3.7 (s, 3H), 4.5 (m, 1H), 5.01 (d, 1H), 6.66 (m, 2H), 6.84 (m, 4H), 7.0 (m, 2H); m/z^{M+1} 387.2

### Step: IV

### Synthesis of 2-[(tert-butoxycarbonyl)amino]-3-{4-[4-(pyridin-2-ylamino)phenoxy]phenyl}propanoate (prepared according to the procedure given in the Example 1, step III)

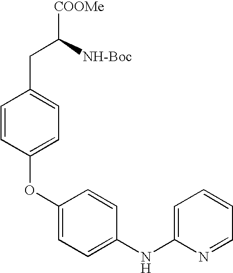

Yield (0.6 g, 54.5%) ¹HNMR. [DMSO-d₆, 400 MHz] δ ppm: 1.42 (s, 9H), 3.0 (m, 2H), 3.7 (s, 3H), 4.5 (d, 1H), 4.99 (d, 1H) 6.45 (s, 1H), 6.7 (m, 2H), 6.92 (d, 2H), 6.99 (d, 2H), 7.07 (d, 2H), 7.31 (d, 2H), 7.49 (t, 1H), 8.1 (d, 1H); m/z^{M+1} 464.2

### Step: V

### Synthesis of Methyl 2-amino-3-{4-[4-(pyridin-2-ylamino)phenoxy]phenyl}propanoate dihydrochloride (prepared according to the procedure given in the Example 1, step-IV)

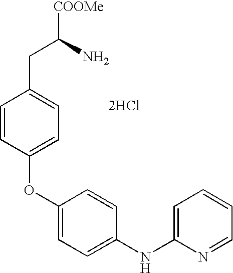

Yield (0.400 g, 85.1%), ¹HNMR. [DMSO-d₆, 400 MHz] δ ppm: 3.11 (m, 2H), 3.7 (s, 3H), 4.2 (t, 1H), 6.9 (m, 3H), 7.1 (m, 3H), 7.2 (d, 2H), 7.48 (d, 2H), 7.96 (m, 2H), 8.63 (bs, 2H), 10.43 (bs, 1H); m/z^{M+1} 364.1

The following compounds were prepared according to the procedure given in example 2.

| Example | | |
|---|---|---|
| No. | Structure | Analytical Data |
| 3 | 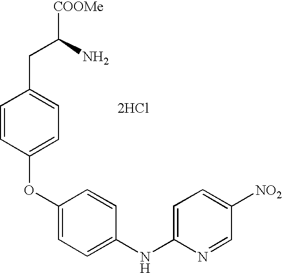 | ¹HNMR. [DMSO-d₆, 400 MHz] δ (ppm):3.1 (m, 2 H), 3.7 (s, 3 H), 4.28 (m, 1 H), 6.94 (m,3 H), 7.03 (d, 2 H), 7.23 (d, 2 H), 7.74 (d, 2 H),8.28 (d, 1 H), 8.53 (bs, 2 H), 9.01 (s, 1 H),10.31 (s, 1 H); m/z^{M+1} 409.2 |
| 4 | 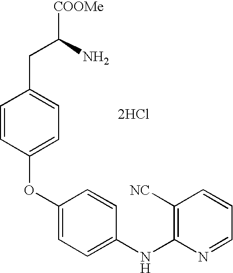 | 1HNMR. [DMSO-d₆, 400 MHz] δ (ppm):3.1 (dd, 2 H), 3.7 (s, 3 H), 4.29 (m, 1 H), 6.9 (m,1 H), 6.96 (m, 4 H), 7.24 (d, 2 H), 7.57 (d, 2 H),8.08 (d, 1 H), 8.34 (d, 1 H), 8.5 (bs, 2 H) 9.13 (s,1 H); m/z^{M+1} 389.1 |
| 5 | 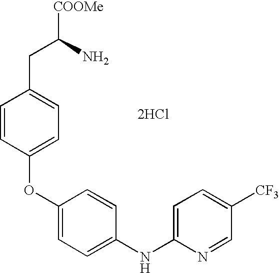 | ¹HNMR. [DMSO-d₆, 400 MHz] δ (ppm):3.1 (m, 2 H), 3.7 (s, 3 H), 4.28 (m, 1 H), 6.94 (m,3 H), 7.0 (d, 2 H), 7.23 (d, 2 H), 7.7 (d, 2 H),7.83 (dd, 1 H), 8.45 (s, 1 H) 8.5 (bs, 2 H), 9.71 (s,1 H); m/z^{M+1} 432.2 |
| 6 | 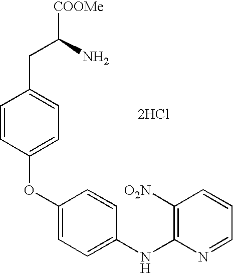 | ¹HNMR. [DMSO-d₆, 400 MHz] δ (ppm):3.11 (m, 2 H), 3.71 (s, 3 H), 4.29 (m, 1 H),6.98 (m, 5 H), 7.24 (d, 2 H), 7.63 (d, 2 H),8.50 (m, 4 H), 9.94 (s, 1 H); m/z^{M+1} 408.9 |
| 7 | 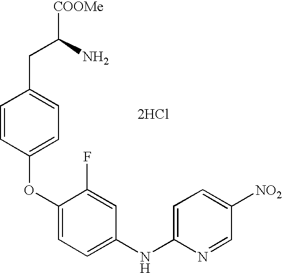 | ¹HNMR. [DMSO-d₆, 400 MHz] δ (ppm):3.09 (m, 2 H), 3.69 (s, 3 H), 4.28 (m, 1 H),6.91 (d, 2 H), 7.01 (d, 1 H), 7.21 (m, 3 H),7.48 (d, 1 H), 8.02 (dd, 1 H), 8.32 (dd, 1 H),8.53 (bs, 2 H), 9.07 (d, 1 H), 10.61 (s, 1 H);m/z^{M+1} 427.1 |
| 8 | 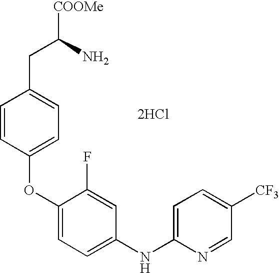 | ¹HNMR. [DMSO-d₆, 400 MHz] δ (ppm):3.05 (m, 2 H), 3.7 (s, 3 H), 4.37 (m, 1 H), 6.89(d, 2 H), 6.96 (d, 1 H), 7.20 (m, 3 H), 7.38 (m,1 H), 7.89 (dd, 1 H), 8.0 (dd, 1 H) 8.39 (bs,2 H), 8.53 (s, 1 H), 9.88 (s, 1 H); m/z^{M+1} 450.1 |
| 9 | 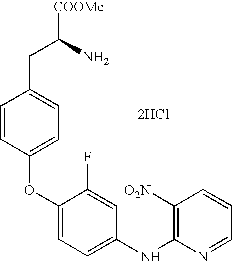 | ¹HNMR. [DMSO-d₆, 400 MHz] δ (ppm):3.11 (m, 2 H), 3.69 (s, 3 H), 4.4 (m, 1 H),6.94 (d, 2 H), 7.0 (dd, 1 H), 7.19 (m, 1 H), 7.24(m, 2 H), 7.48 (dd, 1 H), 7.9 (dd, 1 H), 8.5 (m,2 H) 8.65 (bs, 2 H), 10.0 (s, 1 H); m/z^{M+1} 427.1 |
| 10 | 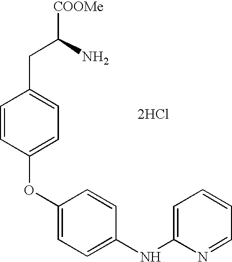 | ¹HNMR. [DMSO-d₆, 400 MHz] δ (ppm):3.14 (m, 2 H), 4.18 (m, 1 H), 6.94 (m, 2 H),7.08 (m, 2 H), 7.11 (m, 2 H), 7.31 (d, 2 H),7.50 (d, 2 H), 7.89 (m, 1 H) 8.02 (d, 1 H), 8.41(bs, 2 H), 10.3 (bs, 1 H); m/z^{M+1} 350.1 |

### Protocols for Biological Testing

### Glucose Uptake Assay Using 3T3-L1 Cells

3T3-L1 cells were differentiated by the addition of differentiation cocktail (72 μg/ml insulin, 0.5 mM IBMX, 400 ng/ml Dexamethasone) for 4 days and were later fed with media without the differentation cocktail for 7-8 days. After differentiation the cells were incubated with the either the reference compound BLX-1002 or with the compounds listed in the table 1 at 1 μM concentrations for 72 hours, and the glucose uptake assay was carried out for 10 minutes by the addition of KRP buffer supplemented with 2.5 μCi/ml ¹⁴C deoxy glucose. Stimulation Index is defined as the amount of ¹⁴C Deoxyglucose uptake induced by 1 μM of BLX-1002 incubated for 72 hours in an assay condition as per the protocol described above with differentiated 3T3-L1 adipocytes. The values for compounds mentioned in table-1 are with reference to the stimulation index of reference compound BLX-1002.

TABLE 1
Effect of compounds on glucose uptake assay in 3T3-L1 cells

| Example No | Stimulation Index |
|---|---|
| BLX-1002 | 1.00 |
| 1 | 1.10 |

### Antidiabetic Activity in Streptozotocin Induced Diabetic Mice

Female Swiss albino mice, at the age of 10 weeks were used in the study. Diabetes was induced in the animals by injecting streptozotocin by i.p. route at a dose of 200 mg/kg body weight. 48 hours after streptozotocin administration, the animals were kept fasting for 6 hours. Subsequently blood was collected, plasma separated and the glucose was estimated. Animals showing greater than 200 mg/dl glucose levels were considered as diabetic and these animals were randomly distributed into various groups. The example 2 listed in the table 2 was administered at a dose of 50-mg/kg body weight by oral route for 7 days. Later the animals were fasted for 6 hours, the blood was collected and the plasma was separated. Biochemical estimations like glucose, cholesterol and triglycerides were carried out using the plasma. The effect of the compounds mentioned in the table was expressed in terms of percentage reduction in biochemical values as compared to the control group. The results are as shown in the table 2.

TABLE 2
Effect of compounds in streptozotocin induced diabetic mice model

| | |
|---|---|
| Example | % Reduction |

| No | Glucose | Triglyceride |
|---|---|---|
| 2 | 52.4 | 49.6 |

We claim:

1. Novel heterocyclic derivatives of the general formula (I) 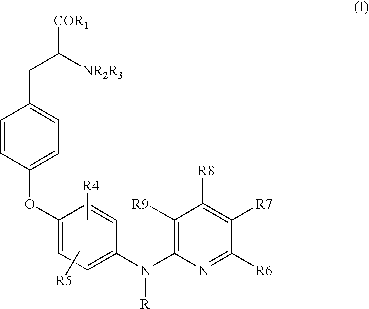
their pharmaceutically acceptable salts, and their pharmaceutical compositions; wherein R represents hydrogen, C₁-C₄ allyl, aryl groups such as phenyl, naphthyl and the like; R₁ represents —OR¹⁰ where R¹⁰ represents hydrogen, substituted or unsubstituted groups selected from C₁-C₄ alkyl or a counter ion, NR¹¹R¹², where R¹¹ and R¹² may be same or different and independently represent H, substituted or unsubstituted groups selected from C₁-C₄ alkyl, aryl groups such as phenyl, naphthyl and the like, heteroaryl groups; R₂ and R₃ may be same or different and independently represent H, COR¹³, substituted or unsubstituted groups selected from C₁-C₄ allyl; where R¹³ represents substituted or unsubstituted groups selected from C₁-C₄ allyl, aryl groups such as phenyl, naphthyl and the like, heteroaryl, aryloxy, alkoxy or aralkoxy groups; R₄ and R₅, may be same or different and independently represent hydrogen, halogen, hydroxy, nitro, cyano, formyl, amino, C₁-C₄ allyl, haloalkyl, alkoxy groups; R₆, R₇, R₈ and R₉ may be same or different and independently represents hydrogen, nitro, cyano, hydroxy, formyl, azido, halo, or substituted or unsubstituted groups selected from C₁-C₄ alkyl, alkoxy, acyl, haloalkyl, amino, hydrazine, monoalkylamino, dialkylamino, acylamino, alkylsulfonyl, alkylsulfinyl, arylsulfonyl, arylsulfinyl, alkylthio, arylthio, alkoxycarbonyl, aryloxycarbonyl, alkoxyalkyl, sulfamoyl, carboxylic acid or its derivatives.

2. Novel Heterocyclic derivatives as claimed in claim 1, are selected from a group comprising of:
I) Methyl-2-amino-3-{4-[4-(pyridin-2-ylamino)phenoxy]phenyl}propanoate dihydrochloride;
II) Methyl (2S)-2-amino-3-{4-[4-(pyridin-2-ylamino)phenoxy]phenyl}propanoate dihydrochloride;
III) Methyl (2S)-2-amino-3-(4-{4-[(5-nitropyridin-2-yl)amino]phenoxy}phenyl) propanoate dihydrochloride;
IV) Methyl (2S)-2-amino-3-(4-{4-[(3-cyanopyridin-2-yl)amino]phenoxy}phenyl)propanoate dihydrochloride;
V) Methyl (2S)-2-amino-3-(4-{4-[(5-trifluoromethyl)pyridin-2-yl)amino]phenoxy}phenyl)propanoate dihydrochloride;
VI) Methyl (2S)-2-amino-3-(4-{4-[(3-nitropyridin-2-yl)amino]phenoxy}phenyl)propanoate dihydrochloride;
VII) Methyl (2S)-2-amino-3-(4-{2-fluoro-4-[(5-nitropyridin-2-yl)amino]phenoxy}phenyl)propanoate dihydrochloride;
VIII) Methyl (2S)-2-amino-3-(4-{2-fluoro-4-[(5-trifluoromethyl)pyridin-2-yl)amino]phenoxy}phenyl)propanoate dihydrochloride;
IX) Methyl (2S)-2-amino-3-(4-{2-fluoro-4-[(3-nitropyridin-2-yl)amino]phenoxy}phenyl)propanoate dihydrochloride;
X) (2S)-2-Amino-3-{4-[4-(pyridin-2-ylamino)phenoxy]phenyl}propanoic acid dihydrochloride.

3. The compound as claimed in claim 1, wherein the said pharmaceutically acceptable salt is selected from the group consisting of hydrochloride, hydrobromide, sodium, potassium or magnesium.

4. A pharmaceutical composition, which comprises of a pharmaceutically effective amount of a novel heterocyclic derivative of formula (I) 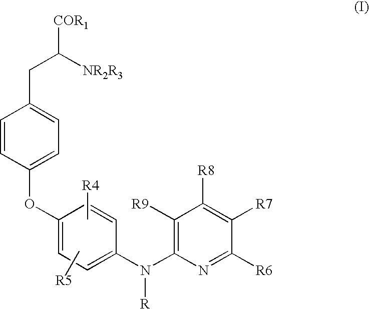
as defined in claim 1 and a pharmaceutically acceptable carrier, diluent, excipient or solvate.

5. A pharmaceutical composition as claimed in claim 4, in the form of a tablet, capsule, powder, syrup, solution, aerosol or suspension.

6. A pharmaceutical composition as claimed in claim 4, wherein the amount of the compound of claim 1 in the composition is less than 60% by weight.

7. A method for reducing blood glucose, free fatty acids, cholesterol, triglycerides levels in the plasma comprising administration of an effective amount of a compound of formula (I) as defined in claim 1 to a patient in need thereof.

8. A method for treating obesity, autoimmune diseases, inflammation, immunological diseases, and cancer disease comprising administration of an effective amount of a compound of formula (I) as defined in claim 1 to a patient in need thereof.

9. A method for treating a disorder associated with insulin resistance comprising administration of an effective amount of a compound of formula (I) as defined in claim 1 to a patient in need thereof.

10. A method for reducing blood glucose levels in the plasma without adipogenic potential comprising administration of an effective amount of a compound as claimed in claim 1 or a compound as claimed in claim 2 to a mammal in need thereof.

11. A method for reducing TNF alfa, IL-6 and IL-beta comprising administration of an effective amount of a compound as claimed in claim 1 or a compound as claimed in claim 2 to a mammal in need thereof.

12. A method for reducing cancer cell progression comprising administration of an effective amount of a compound as claimed in claim 1 or a compound as claimed in claim 2 to a mammal in need thereof.
